(19) United States
(12) Patent Application Publication
(10) Pub. No.: US 2023/0150446 A1
(43) Pub. Date: May 18, 2023

(54) Fire Safety Seat Belt

(71) Applicant: Bonnie Koger, Dayton, OH (US)

(72) Inventor: Bonnie Koger, Dayton, OH (US)

(21) Appl. No.: 17/527,389

(22) Filed: Nov. 16, 2021

(51) Int. Cl. B60R 21/01; B60R 22/32; B60Q 9/00; E05B 77/54; E05B 81/56

(52) U.S. Cl. CPC B60R 21/01; B60R 22/321; B60Q 9/00; E05B 77/54; E05B 81/56; B60R 2021/01034; B60R 2021/01265; G01N 33/0036

(57) ABSTRACT

Ingenious and practical, the Fire Safety Seat Belt has sensors around the car to tell the vehicle's computer to unlock the seat belts. When picking out your vehicle and wanting it fully loaded, drivers would definitely want this one extra safety mechanism, the Fire Safety Seat Belt. Not only would this device sense fires but is designed to sense the presence of excess water. This apparatus once sensing the vehicle is either on fire or flooding would release the seat belts and unlock the doors. This process would be lifesaving, allowing the individuals to easily get out of the vehicle and harm's way and to safety either by their own means or with aid of a good Samaritan. With the knowledge that you would be safe wearing your seat belt, without the potential of being stuck in the vehicle during an emergency, would also ease people's fears. Thus, this would help more individual's to make the choice to buckle up and potentially save more lives.

### BACKGROUND

In most places it's the law to buckle up. Doing so usually saves lives, countless lives have been saved because vehicle occupants wear a seat belt. There are, however, some incidents when a seat belt was a liability due to the individual not being able to get out of their seat belt before further disaster occurred. Some individuals feel that because of this it is their personal choice to not wear their seat belt for fear of being stuck in case of emergency. Fear of not been able to get out of their car after a wreck or the vehicle being on fire because they couldn't get out of the seat belt is real. Cases occur of someone helping not being able to get occupants out of a vehicle because it was burning and the seat belt was in the way. There have been no products available as original equipment or as an aftermarket to address this problem.

An apparatus or system designed to prevent individual's from being trapped by their seatbelts and locked doors in an emergency situation. There have been no products available as original equipment or as an aftermarket to address this problem either.

There exists a need for a device and a system such as the Fire Safety Seat bealt, that is not being met by any known or disclosed device or system of present.

### SUMMARY OF THE INVENTION

The present invention overcomes the above referred to difficulties in a convenient, simple and efficient manner. The Fire Safety Seat belt is uniquely designed with self-adhesive sensors specifically designed to check for fire, smoke, or flooding. Once triggered there is a relay to the vehicle to release the seat belts and unlock the doors for easy exiting of the vehicle. Thus this apparatus and system is essential in helping to save lives and allow individual's the knowledge of their safe removal from their seat belts in an emergency situation.

A vehicle safety system comprising a sensor for indicating a loss in a cabin life support parameter of an enclosed cabin of the vehicle of a predetermined amount. The system also includes a latch release mechanism for a mechanical feature of the vehicle triggered by the sensor. The system further includes a circuit for a control of the sensor and the latch mechanism and a communication there between.

The vehicle safety system comprises a failsafe based on a sensor reading difference of a cabin life support parameter greater than twenty percent for a combination of oxygen content, cabin pressure and temperature from an initial cabin environment.

### BRIEF DESCRIPTION OF THE DRAWINGS

FIG. 1 is a front perspective view of the Fire Safety Seat Belt showing: seat belt referenced as A., automatic unlocking mechanism (when high heat detected) referenced as B., thin adhesive heat sensor referenced as C., in accordance with an embodiment of the present disclosure.

FIG. 2 is a perspective detail view of a vehicle door latch controlled by the disclosure in accordance with an embodiment of the present disclosure.

FIG. 3 is a flow diagram of steps to unlatching a seat belt and a door latch based on a cabin pressure in the vehicle in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

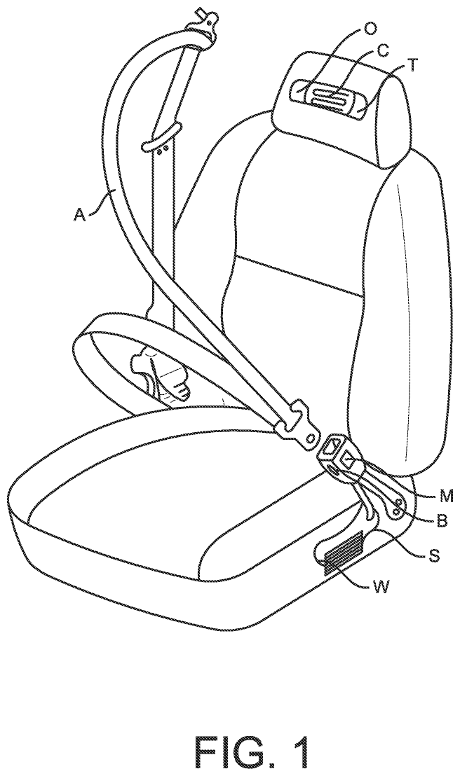

FIG. 1 is a front perspective view of the Fire Safety Seat Belt showing: seat belt referenced as A., automatic unlocking mechanism (when high heat detected) referenced as B., thin adhesive heat sensor referenced as C., oxygen sensor O, pressure sensors S, microprocessors or state machine circuits M, transceivers T and wires W in accordance with an embodiment of the present disclosure.

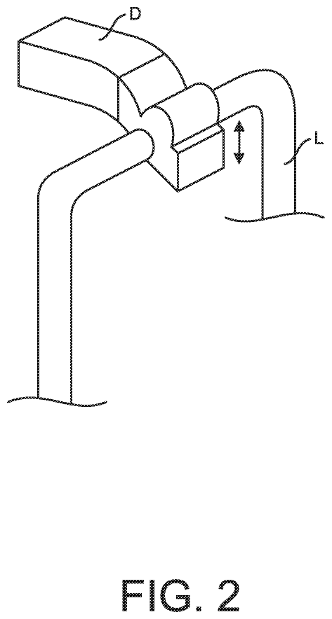

FIG. 2 is a perspective detail view of a vehicle door latch controlled by the disclosure in accordance with an embodiment of the present disclosure. The detail view includes a door latch L and a locking or latching lever D which is released from the latch L in accordance with the sensors and circuits of the disclosure.

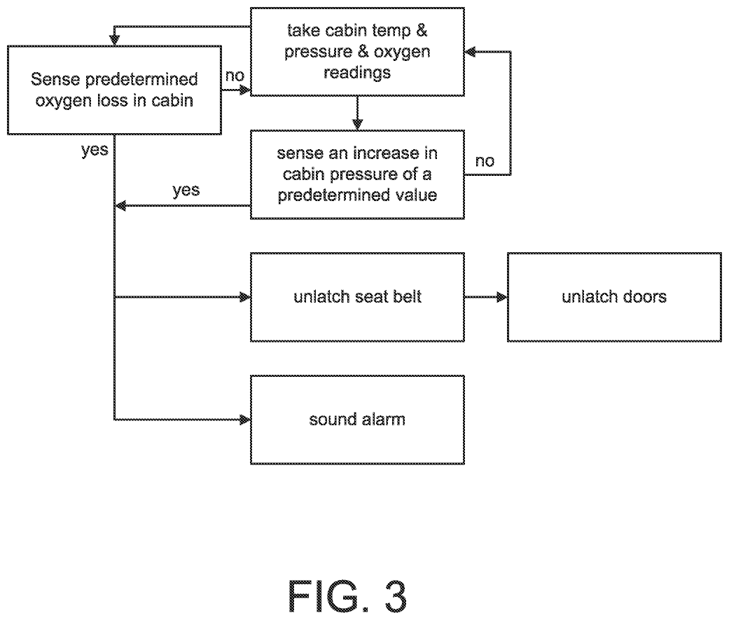

FIG. 3 is a flow diagram of steps to unlatching a seat belt and a door latch based on a cabin pressure in the vehicle in accordance with an embodiment of the present disclosure. First an initial cabin pressure is read through the pressure sensor(s) and logged into the microprocessor circuits of the disclosure.

Based on a predetermined increase of cabin pressure which in embodiments is approximately 450 Pascals, the control circuits unlatch the seat belt and the door latch and sound an alarm to protect and notify those in jeopardy and care givers etc. A 450 Pascal increase in cabin pressure is meant to indicate a fire or a flooding of the vehicle cabin. A loss of oxygen of 20 percent from an initial reading of the sensor O also indicates a fire or a flood of water etc that endangers occupants and results in action taken by the disclosure for safety.

The Fire Safety Seatbelts come with self-adhesive sensors patches. These sensors are specially designed to sense temperature that could start a flame, smoke or excessive water in the vehicle. When any sensor is triggered it is relayed to the vehicle's circuit to unlock the seatbelt and vehicle doors. This allows the passenger or driver to escape or be removed from the vehicle with ease.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

What is claimed is:

1. A vehicle safety system comprising:
a sensor for indicating a loss in a cabin life support parameter of an enclosed cabin of the vehicle of a predetermined amount;
a latch release mechanism for a mechanical feature of the vehicle triggered by the sensor; and
a circuit for a control of the sensor and the latch mechanism and a communication there between.

2. The vehicle safety system of claim 1, wherein the cabin life support paras is a cabin pressure.

3. The vehicle safety system of claim 1, wherein the predetermined amount is a 450 Pascal drop in a cabin pressure of the vehicle.

4. The vehicle safety system of claim 1, wherein the cabin life support parameter is a heat content thereof.

5. The vehicle safety system of claim 1, wherein the predetermined amount is an oxygen content drop exceeding twenty percent.

6. The vehicle safety system of claim 1, wherein the predetermined amount is a temperature reading from the sensor exceeding 300 degrees Fahrenheit.

7. The vehicle safety system of claim 1, wherein the cabin life support parameter is an oxygen content in an enclosed cabin of the vehicle.

8. The vehicle safety system of claim 1, wherein the latch release mechanism is a seat belt latch.

9. The vehicle safety system of claim 1, wherein the latch release mechanism is a vehicle door latch.

10. The vehicle safety system of claim 1, wherein the circuit is a state machine circuit comprising logic circuits and wired communication therein.

11. The vehicle safety system of claim 1, wherein the circuit and communication there between the sensor and the latch mechanism further comprises a wireless transceiver circuit.

12. The vehicle safety system of claim 1, wherein the sensor is an oxygen content sensor.

13. The vehicle safety system of claim 1, wherein the sensor is a cabin pressure sensor.

14. The vehicle safety system of claim 1, wherein the sensor is a temperature sensor.

15. The vehicle safety system of claim 1, further comprising a vehicle alarm system triggered by the sensor.

16. The vehicle safety system of claim 1, further comprising a memory configured to store initial cabin pressure, temperature and oxygen content taken by the sensor.

17. The vehicle safety system of claim 1, further comprising a sensor reading difference of a cabin life support parameter greater than twenty percent for a combination of oxygen content, cabin pressure and temperature from an initial cabin environment.
